(19) United States
(12) Patent Application Publication
(10) Pub. No.: US 2025/0303054 A1
(43) Pub. Date: Oct. 2, 2025

(54) Dual-Channel Spike for Intravenous Fluid Bag

(71) Applicant: Profound Medical Inc., Mississauga (CA)

(72) Inventors: Nicholas Alexander Lavdas, London (CA); Hooman Reza Zadeh Tabatabai, Richmond Hill (CA); Christopher Jerger, Toronto (CA); Christopher Driver, Oakville (CA)

(21) Appl. No.: 19/092,176

(22) Filed: Mar. 27, 2025

Related U.S. Application Data
(60) Provisional application No. 63/570,533, filed on Mar. 27, 2024.

(51) Int. Cl. A61M 5/14; A61F 7/00; A61F 7/12; A61M 5/162; A61N 7/02

(52) U.S. Cl. CPC A61M 5/1409; A61F 7/12; A61M 5/162; A61N 7/02; A61F 2007/0028; A61F 2007/0063; A61M 2205/3606; A61M 2205/3626

(57) ABSTRACT

A dual-channel spike includes a shaft having inlet and outlet openings defined therein, a pointed distal end, and a proximal end, the shaft extending along an axis between the proximal end and the pointed distal end, the pointed distal end configured to puncture an intravenous fluid bag; a handle attached to the proximal end of the shaft; an inlet connector fluidly coupled to the handle, the inlet connector configured to be fluidly connected to a first fluid line; an outlet connector fluidly coupled to the handle, the outlet connector configured to be fluidly connected to a second fluid line; an inlet channel defined in the shaft and the handle, the inlet channel fluidly coupling the inlet connector and the outlet opening; and an outlet channel defined in the shaft and the handle, the outlet channel fluidly coupling the outlet connector and the inlet opening.

### CROSS-REFERENCE TO RELATED APPLICATIONS

This application claims priority to U.S. Provisional Application No. 63/570,533, titled “Dual-Channel Spike For Intravenous Fluid Bag,” filed on Mar. 27, 2024, which is hereby incorporated by reference.

### TECHNICAL FIELD

This application relates generally to fluid connectors for thermal therapy systems.

### BACKGROUND

Intravenous (IV) fluid bags can be spiked to insert or remove liquid. Conventional IV spikes include a single channel that allows for one-way fluid flow. For example, the fluid can exit through the channel in the IV spike to an IV fluid line that is connected to a patient. In another example, medicine can be added to the IV fluid through the channel in the IV spike.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a medical system comprising a medical device having a cooling channel defined therein; an intravenous (IV) fluid bag holding a fluid; a dual-channel spike disposed in the IV fluid bag, the dual-channel spike defining first and second channels that are fluidly connected to the IV fluid bag, the dual-channel spike having a pointed distal end configured to puncture the IV fluid bag; a first line fluidly connected to the first channel and to an inlet of the cooling channel; and a second line fluidly connected to the second channel and to an outlet of the cooling channel, whereby the fluid can be recirculated between the IV fluid bag and the cooling channel using the first and second channels in the dual-channel spike.

In one or more embodiments, the medical system further comprises a first pump fluidly coupled to the first line; and/or a second pump fluidly coupled to the second line. In one or more embodiments, the dual-channel spike includes a shaft having proximal and distal ends, first and second tubes attached to the proximal end of the shaft, a first opening defined in the shaft, and a second opening defined in the shaft, wherein the first line is fluidly connected to the first tube, the second line is fluidly connected to the second tube, the first channel is fluidly coupled to the first opening and the first tube, and the second channel is fluidly coupled to the second opening and the second tube.

In one or more embodiments, the first and second openings are positionally offset with respect to an axis of the shaft, the shaft extending along the axis. In one or more embodiments, the first and second openings are offset with respect to a circumference of the shaft. In one or more embodiments, the second opening is defined at or in the distal end of the shaft. In one or more embodiments, the pointed distal end is located at the distal end of the shaft.

In one or more embodiments, the medical device comprises an ultrasound applicator or an endorectal cooling device.

Another aspect of the invention is directed to a dual-channel spike comprising a shaft having inlet and outlet openings defined therein, a pointed distal end, and a proximal end, the shaft extending along an axis between the proximal end and the pointed distal end, the pointed distal end configured to puncture an intravenous fluid bag; a handle attached to the proximal end of the shaft; an inlet connector fluidly coupled to the handle, the inlet connector configured to be fluidly connected to a first fluid line; an outlet connector fluidly coupled to the handle, the outlet connector configured to be fluidly connected to a second fluid line; an inlet channel defined in the shaft and the handle, the inlet channel fluidly coupling the inlet connector and the outlet opening; and an outlet channel defined in the shaft and the handle, the outlet channel fluidly coupling the outlet connector and the inlet opening.

In one or more embodiments, the inlet and outlet openings are positionally offset along the shaft with respect to the axis. In one or more embodiments, the inlet opening is closer to the handle than the outlet opening. In one or more embodiments, the inlet and outlet openings are offset with respect to a circumference of the shaft. In one or more embodiments, the outlet opening is defined at or in the pointed distal end of the shaft.

In one or more embodiments, the axis is a first axis, the shaft has a width that is measured with respect to a second axis that is orthogonal to the first axis, and the pointed distal end has an angled planar face that is defined by a plane, the plane defined by (a) a third axis that is orthogonal to the first and second axes and (b) a fourth axis that is disposed at an angle relative to the first axis, the angle greater than or equal to about 30 degrees and less than or equal to about 80 degrees.

In one or more embodiments, the dual-channel spike further comprises inlet and outlet tubes attached to a proximal end of the handle, the inlet and outlet connectors defined in the inlet and outlet tubes, respectively. In one or more embodiments, the axis is a first axis, the inlet tube extends along a second axis, the outlet tube extends along a third axis, and the second axis is disposed at an angle relative to the third axis, the angle greater than or equal to about 15 degrees and less than or equal to about 45 degrees.

In one or more embodiments, the axis is a first axis, and a distal end of the handle has a smaller width than the proximal end of the handle, the width measured with respect to a second axis that is orthogonal to the first axis. In one or more embodiments, the dual-channel spike further comprises an over-mold covering at least an external surface of the shaft. In one or more embodiments, the dual-channel spike further comprises a barb or an O-ring disposed on the shaft, the barb or the O-ring configured to mechanically engage the IV fluid bag to improve a fluid seal between the dual-channel spike and the IV fluid bag.

Another aspect of the invention is directed to a kit comprising an intravenous (IV) fluid bag; and a dual-channel spike. The dual-channel spike comprises a shaft having inlet and outlet openings defined therein, a pointed distal end, and a proximal end, the shaft extending along an axis between the proximal end and the pointed distal end, the pointed distal end configured to puncture the IV fluid bag; a handle attached to the proximal end of the shaft; an inlet connector fluidly coupled to the handle, the inlet connector configured to be fluidly connected to a first fluid line; an outlet connector fluidly coupled to the handle, the outlet connector configured to be fluidly connected to a second fluid line; an inlet outlet channel defined in the shaft and the handle, the inlet channel fluidly coupling the inlet connector and the outlet opening; and an outlet channel defined in the shaft and the handle, the outlet channel fluidly coupling the outlet connector and the inlet opening.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a diagram of a medical system according to one or more embodiments.

FIG. 2A is a simplified and partially transparent illustration of an ultrasound applicator according to one or more embodiments.

FIG. 2B is a simplified and partially transparent illustration of an endorectal cooling device according to one or more embodiments.

FIG. 3 is a side view of the dual-channel spike according to one or more embodiments.

FIG. 4A is a cross section of the dual-channel spike shown in FIG. 3, according to one or more embodiments.

FIG. 4B is a cross section of the dual-channel spike shown in FIG. 3 inserted into an intravenous (IV) fluid bag.

FIG. 5 is an isometric perspective view of the cross section shown in FIG. 4A according to one or more embodiments.

FIGS. 6 and 7 are isometric views of the dual-channel spike shown in FIG. 3 according to one or more embodiments.

FIG. 8 is an isometric view of the shaft of the dual-channel spike according to one or more embodiments.

FIG. 9 is an isometric view of the shaft of the dual-channel spike according to one or more embodiments.

FIG. 10 is a side view of a proximal portion of the dual-channel spike according to one or more embodiments.

FIG. 11 is an isometric perspective view of a proximal portion of the dual-channel spike according to one or more embodiments.

FIG. 12 shows an example dual-channel spike inserted into an IV fluid bag according to one or more embodiments.

FIG. 13 is a block diagram of a kit according to one or more embodiments.

FIG. 14 is a flow chart of a method for recirculating cooling fluid using a dual-channel spike according to one or more embodiments.

### DETAILED DESCRIPTION

A dual-channel intravenous (IV) spike for an IV fluid bag is configured to allow cooling fluid to recirculate between the IV fluid bag and another location, such as a medical device or a second bag such as a second IV fluid bag or a larger storage bag. An example of a medical device is an ultrasound probe or an endorectal cooling device (ECD). The fluid can comprise saline, a cooling fluid, aqueous solutions (e.g., that include drugs, contrast, or another solute). The second bag can include drugs, contrast, or another substance to be combined or mixed with the IV fluid bag. Alternatively, the second bag can include saline, for example a larger volume of saline used to fill an IV fluid bag.

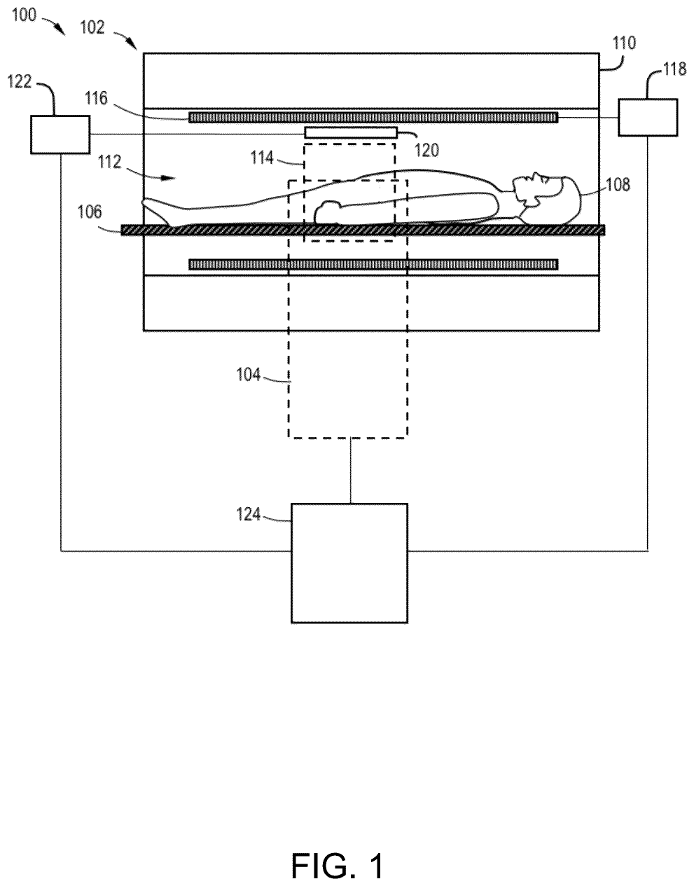

FIG. 1 is a diagram of a medical system 100 in which at least some of the apparatus, systems, and/or methods disclosed herein can be employed, in accordance with at least some embodiments. The system 100 includes a patient support 106 (on which a patient 108 is shown), a magnetic resonance imaging (MRI) system 102 and an image-guided energy delivery system 104.

The magnetic resonance system 102 includes a magnet 110 disposed about an opening 112, an imaging zone 114 in which the magnetic field is strong and uniform enough to perform MRI, a set of magnetic field gradient coils 116 to change the magnetic field rapidly to enable the spatial coding of MRI signals, a magnetic field gradient coil power supply 118 that supplies current to the magnetic field gradient coils 116 and is controlled as a function of time, a transmit/receive coil 120 (also known as a “body” coil) to manipulate the orientations of magnetic spins within the imaging zone 114, a radio frequency transceiver 122 connected to the transmit/receive coil 120, and a computer 124, which performs tasks (by executing instructions and/or otherwise) to facilitate operation of the MRI system 102 and is coupled to the radio frequency transceiver 122, the magnetic field gradient coil power supply 118, and the image-guided energy delivery system 104. The image-guided energy delivery system 104 includes a therapeutic applicator, such as an ultrasound applicator, to perform image-guided therapy (e.g., thermal therapy) to treat a treatment volume in the patient 108.

The MRI computer 124 can include more than one computer in some embodiments, at least one of which can be dedicated to the MRI system 102. In at least some embodiments, the MRI computer 124 and/or one or more other computing devices (not shown) in and/or coupled to the system 100 may also perform one or more tasks (by executing instructions and/or otherwise) such as to control the driving or operating frequency of the ultrasound elements in the therapeutic applicator, such as at the center frequency (f₀) and/or at a higher harmonic (3f₀) of the center frequency.

One or more of the computers, including computer 124, can include a treatment plan for and/or program instructions for determining a treatment plan (e.g., in real time) for the patient 108 that includes the target treatment volume and the desired or minimal energy (e.g., thermal) dose for the target treatment volume. The treatment plan can also include the desired operating or driving frequency of the ultrasound elements, such as f₀ and/or 3f₀. The computer(s) can use images from the MRI system 102 to image guide the rotational position and insertion-retraction position of the therapeutic applicator. In some embodiments, one or more dedicated computers control the image-guided energy delivery system 104. Some or all of the foregoing computers can be in communication with one another (e.g., over a local area network, a wide area network, a cellular network, a WiFi network, or other network), for example through a software-controlled link to a communication network.

In some embodiments, the treatment plan includes a set of initial parameters for driving each ultrasound element such as its initial frequency, initial phase, and initial amplitude. These parameters can be updated in real time based on the measured temperature of the target volume, for example as determined by MR thermometry.

The magnetic resonance system 102 can be replaced with another imaging system such as an ultrasound imaging system. Alternatively, the image-guided energy delivery system 104 can be used without an imaging system in which case the image-guided energy delivery system 104 can be referred to as an energy delivery system 104.

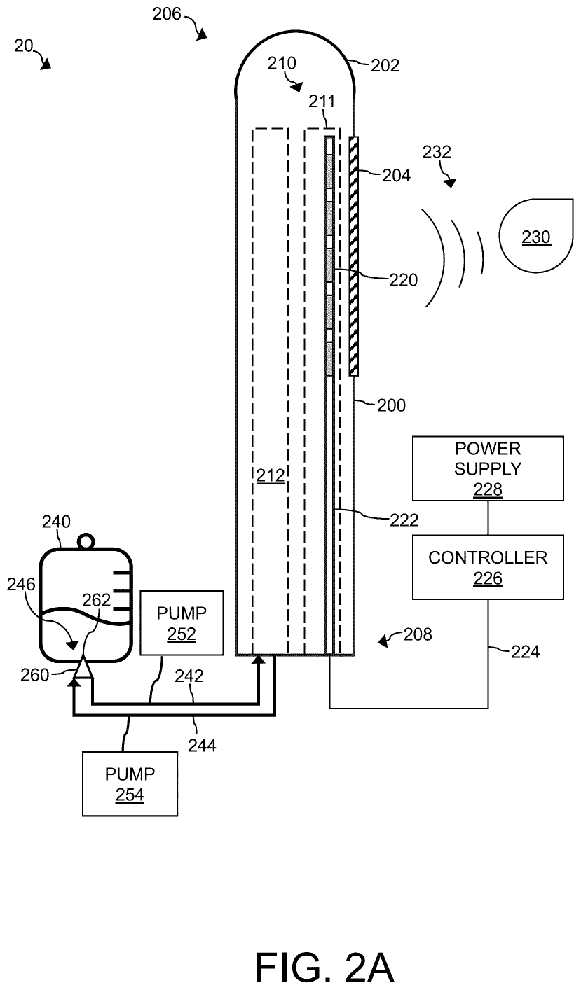

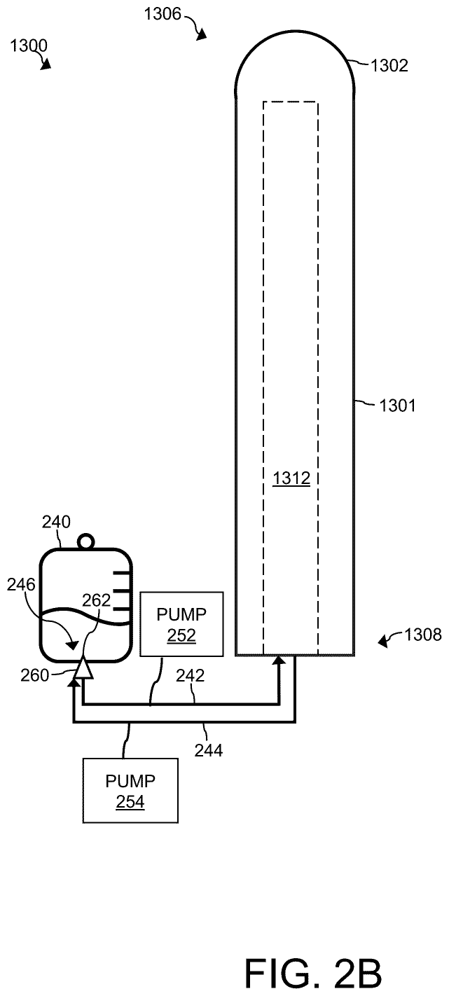

FIG. 2A is a simplified and partially transparent illustration of an ultrasound applicator 20 according to one or more embodiments. The ultrasound applicator 20 can be a therapeutic applicator for an image-guided energy delivery system 104 or an energy delivery system 104 (FIG. 1). The ultrasound applicator 20 includes a shaft 200 attached to or including a tip 202 at a distal end 206 of the shaft 200.

Multiple channels 210 can be defined in the shaft 200 of the ultrasound applicator 20. Each channel 210 is defined at a proximal end 208 of the shaft 200 and extends towards the distal end 206 of the shaft 200. For example, an ultrasound channel 211 can be defined in the shaft 200 of the ultrasound applicator 20. The ultrasound channel 211 is configured to receive one or more ultrasound transducers 220. The ultrasound transducer(s) 220 can comprise an array of ultrasound transducers, such as a linear array or a focused array of ultrasound transducers. The ultrasound transducer(s) 220 can be mounted on and/or electrically connected to an elongated circuit board 222. The elongated circuit board 222 can be electrically coupled and/or connected (e.g., via wire(s) 224) to a controller 226 that can selectively provide electrical power, produced by a power supply 228, at a frequency, relative phase, and/or amplitude according to a treatment plan so as to treat a target volume 230 in a patient. The controller 226 and the power supply 228 can be combined in some embodiments. Ultrasound energy 232 produced by the ultrasound transducer(s) 220 can pass through an ultrasound window 204 in the shaft 200 and can be focused, geometrically and/or electronically, onto the target volume 230.

A cooling channel 212 is defined in the shaft 200 of the ultrasound applicator 20. The cooling channel 212 is configured to receive cooling fluid that can be used to cool the ultrasound applicator 20 and/or the surrounding volume (e.g., surrounding tissue) during ultrasound therapy. The cooling fluid can be provided from a cooling fluid reservoir, such as an intravenous (IV) fluid bag 240. For example, the cooling fluid can be recirculated between the IV fluid bag 240 and the cooling channel 212. Cooler (e.g., room temperature) cooling fluid can flow from the IV fluid bag 240 to the cooling channel 212 through an inlet line (e.g., a first line) 242. After passing through at least a portion of the cooling channel 212 and receiving heat from the ultrasound applicator 20, warmer cooling fluid can flow from the cooling channel 212 to the IV fluid bag 240 through an outlet line (e.g., a second line) 244. A pump 252 can be fluidly coupled to the inlet line 242 to drive the cooler cooling fluid into the cooling channel 212 and/or a pump 254 can be fluidly coupled to the outlet line 244 to drive the warmer cooling fluid into the IV fluid bag 240. Alternatively, a pump 252 or 254 can be fluidly coupled to the inlet line 242 and to the outlet line 244 to drive the respective cooling fluid. The inlet line 242 and the outlet line 244 can comprise respective fluid tubes.

A dual-channel spike 260 can be disposed in the IV fluid bag 240 and fluidly connected to the inlet line 242 and to the outlet line 244. The dual-channel spike 260 includes a first channel that is fluidly connected to the inlet line 242 and the IV fluid bag 240 and a second channel that is fluidly connected to the outlet line 244 and the IV fluid bag 240. The dual-channel spike 260 includes a pointed end 262 that is configured to puncture the IV fluid bag 240 (e.g., the bottom of the IV fluid bag 240) and insert the pointed end 262 through a punctured hole 246 in the IV fluid bag 240.

FIG. 2B is a simplified and partially transparent illustration of an ECD 1300 according to one or more embodiments. The ECD 1300 includes a shaft 1301 attached to or including a tip 1302. The shaft 1301 can be curved or bent to conform to a rectal cavity. A cooling channel 1312 is defined in the shaft 1301 of the ECD 1300. The cooling channel 1312 is defined at a proximal end 1308 of the shaft 1301 and extends towards the distal end 1306 of the shaft 1301. The cooling channel 1312 is configured to receive cooling fluid that can be used to cool the ECD 1300 and/or the surrounding volume (e.g., surrounding tissue), for example during thermal therapy. The cooling fluid can be provided from a cooling fluid reservoir, such as an intravenous (IV) fluid bag 240 using a dual-channel spike 260 in the same manner as discussed above with respect to the ultrasound applicator 20.

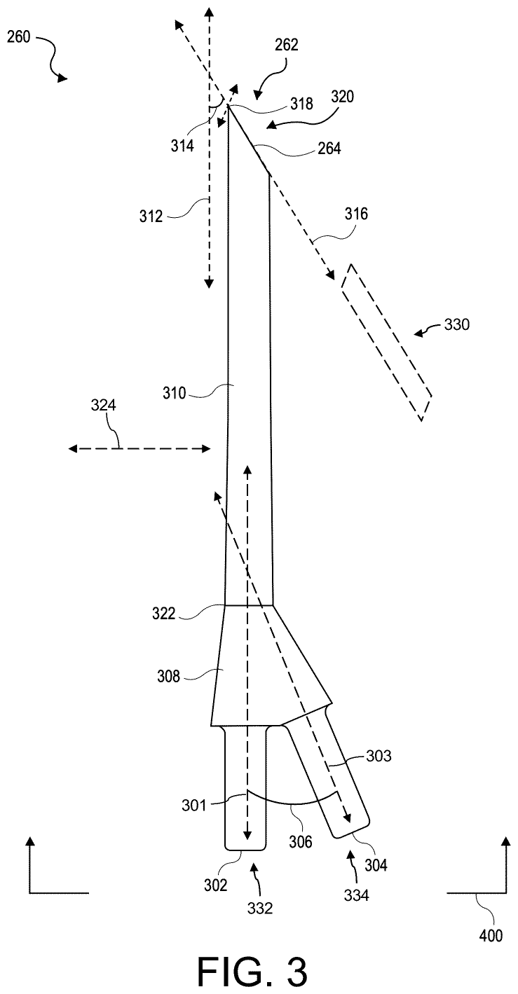

FIG. 3 is a side view of the dual-channel spike 260 according to one or more embodiments. The dual-channel spike 260 includes an outlet tube (e.g., a first tube) 302 and an inlet tube (e.g., a second tube) 304. The outlet tube 302 is configured to be fluidly coupled to the inlet line 242 for example to provide cooler cooling fluid to the inlet line 242 (e.g., which can be fluidly coupled to a medical device such as an ultrasound applicator 20 or an ECD 1300). The inlet tube 304 is configured to be fluidly coupled to the outlet line 244 for example to receive warmer cooling fluid from the outlet line 244 (e.g., which can be fluidly coupled to a medical device such as an ultrasound applicator 20 or an ECD 1300). The outlet tube 302 has an outlet or first opening 332. The inlet tube 304 has an inlet or second opening 334.

The outlet tube 302 and the inlet tube 304 can form a V-shape having or defining an angle 306, which can range from about 15 degrees to about 45 degrees including about 30 degrees and any angle or range between any two of the foregoing values. The angle 306 can be defined between a first axis 301 along which the outlet tube 302 extends and a second axis 303 along which the inlet tube 304 extends. As used herein, “about” means plus or minus 10% of the relevant value. The V-shape can terminate at a transition region or handle 308 (in general handle) where the outlet tube 302 and the inlet tube 304 are fluidly coupled to respective channels in an elongated shaft 310 that includes or is attached to the pointed end 262. In other embodiments, the outlet tube 302 and the inlet tube 304 can be parallel or substantially parallel (e.g., within about 10%) with each other.

The shaft 310 extends parallel to a shaft axis 312. The shaft axis 312 is parallel to the first axis 301. In other embodiments, the shaft axis 312 can be parallel to the second axis 303 and/or to the first and second axes 301, 303. A distal end 320 of the shaft 310 can have an angled face 264 that forms or defines the pointed end 262. The angled face 264 can be defined by an angle 314 formed between an axis 316 that extends parallel to the face 264 and the shaft axis 312. The angled face 264 can be planar and formed with respect to a plane 330 defined by the axis 316 and an axis 318 that is orthogonal to the axis 316 and to the shaft axis 312. The axis 318 extends into and out of the page in FIG. 3. The angle 314 can range from about 30 degrees to about 80 degrees including about 45 degrees, about 60 degrees, about 75 degrees, and any angle or range between any two of the foregoing values. The angle 314 can have another value in other embodiments.

In some embodiments, the shaft 310 can have a tapered outer diameter or width with the distal end or portion (in general, distal end) 320 having a smaller outer diameter (or width) compared to a proximal end or portion (in general, proximal end) 322 of the shaft 310. For example the distal end 320 can have a diameter of about 5.5 mm and the proximal end 322 can have a diameter of about 6 mm. In some embodiments, the tapered outer diameter is with respect to the last 25 mm (e.g., about 20 mm to about 30 mm) of the shaft 310. In some embodiments, the spike 260 can have a maximum outer diameter in the range of about 4 mm to about 6.5 mm including about 4.5 mm, about 5 mm, about 5.5 mm, about 6 mm, and any value or range between any two of the foregoing values. The width or diameter of the shaft 310 can be measured with respect to a lateral axis 324 that is orthogonal to axes 312, 318. Additionally or alternatively, the width or diameter of the shaft 310 can be measured with respect to axis 318.

The spike 260 can be configured to be inserted into an infusion port (or spike port) of an IV fluid bag 240 that is designed and/or configured to accept spikes having an outer diameter in the range of about 4 mm to about 6.5 mm or another outer diameter.

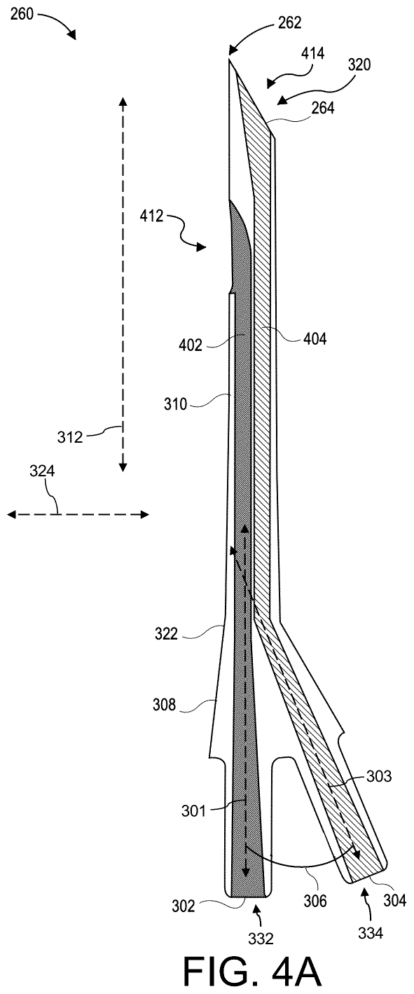

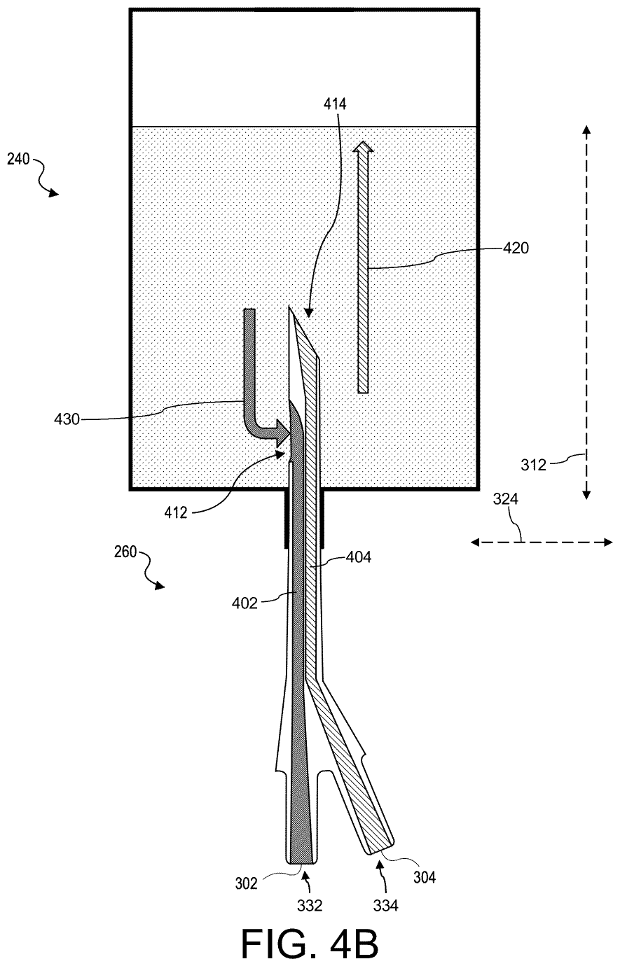

FIG. 4A is a cross section of the dual-channel spike 260 taken through plane 400 in FIG. 3, according to one or more embodiments. The cross section reveals an outlet channel (e.g., a first channel) 402 and an inlet channel (e.g., a second channel) 404. The outlet channel 402 extends between and fluidly couples the outlet tube 302 (e.g., the outlet opening 332) and a first or inlet opening 412 defined in the shaft 310. Fluid flows through the inlet opening 412 into the outlet channel 402 and out through the outlet opening 332 (e.g., to an inlet line 242). The inlet channel 404 extends between and fluidly couples the inlet tube 304 (e.g., the inlet opening 334) and a second or outlet opening 414 defined at the pointed end 262 (e.g., defined in the angled face 264) of the shaft 310. Fluid flows through the inlet opening 334 (e.g., from an outlet line 244) into the inlet channel 404 and out through the outlet opening 414. The inlet and outlet openings 412, 414 can be defined in different locations of the shaft 310 in other embodiments.

The outlet and inlet openings 412, 414 are positionally offset with respect to each other along the shaft 310 (e.g., relative to the shaft axis 312) to reduce the likelihood of a fluid short circuit in which warmer cooling fluid passes out of the outlet opening 414 and flows back into the inlet opening 412 without having time to cool in the IV fluid bag. Additionally or alternatively, the inlet and outlet openings 412, 414 are offset (e.g., angularly offset) with respect to a circumference of the shaft 310. For example, the inlet and outlet openings 412, 414 can be located on opposing sides of the shaft 310 (e.g., angularly offset by about 180 degrees) to further reduce the likelihood of a fluid short circuit. The location of the outlet opening 414 at the pointed end 262 of the dual-channel spike 260 is configured to direct warmer cooling fluid 420 towards the top of the IV fluid bag 240 while the location of the inlet opening 412 lower than the outlet opening 414 (e.g., relative to axis 312) and on the side of the dual-channel spike 260 (e.g., in a direction parallel to axis 322) is configured to receive cooler cooling fluid 430, that has settled towards the bottom of the IV fluid bag 240, into the inlet opening 412, for example as illustrated in FIG. 4B.

In other embodiments, the outlet channel 402 and the inlet opening 412 can be switched with the inlet channel 404 and the outlet opening 414, respectively.

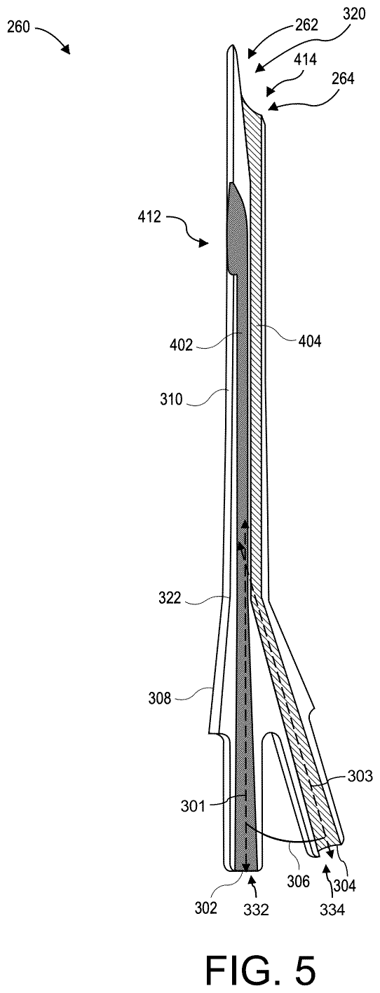

FIG. 5 is an isometric perspective view of the cross section illustrated in FIG. 4A to further illustrate the outlet channel 402, the inlet opening 412, the inlet channel 404, and the outlet opening 414 according to one or more embodiments.

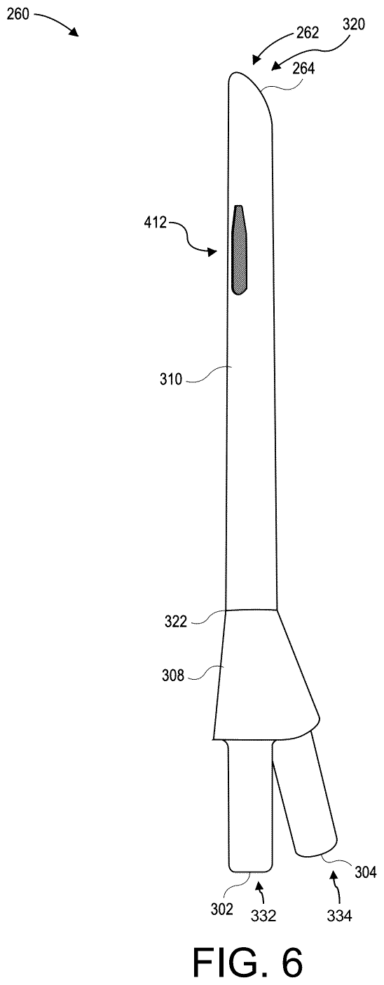

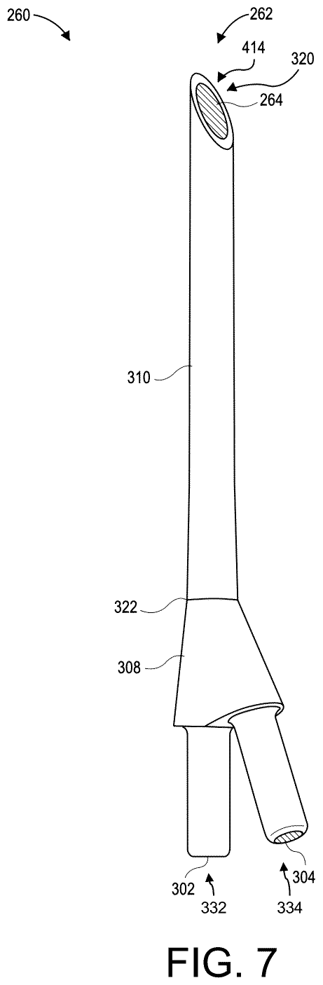

FIGS. 6 and 7 are isometric views of the dual-channel spike 260 to further illustrate the inlet opening 412 and the outlet opening 414, respectively, according to one or more embodiments.

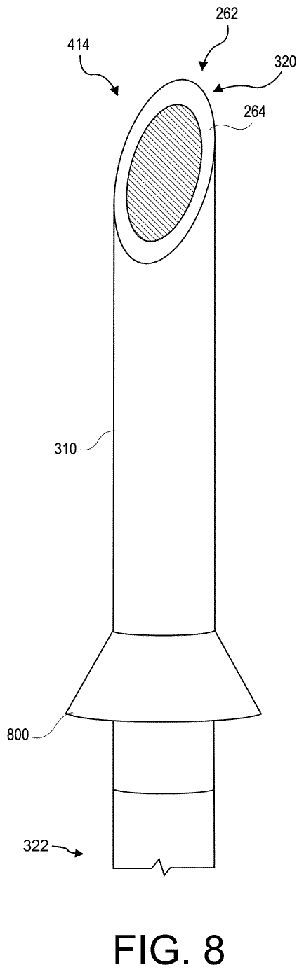

FIG. 8 is an isometric view of the shaft 310 according to one or more embodiments. A barb 800 is disposed on and/or attached to the shaft 310. The barb 800 is configured to mechanically engage an IV fluid bag 240 to increase the force required to pull the dual-channel spike 260 out of the IV fluid bag 240 after insertion to prevent accidental removal. The barb 800 can also reduce water leakage around the shaft 310 and the dual-channel spike 260, such as by improving the fluid seal between the dual-channel spike 260 and the IV fluid bag. The barb 800 can be located at or towards the proximal end 322 of the shaft 310 (e.g., closer to the proximal end 322 than the distal end 320). In one or more alternative embodiments, the barb 800 can be replaced with a flange.

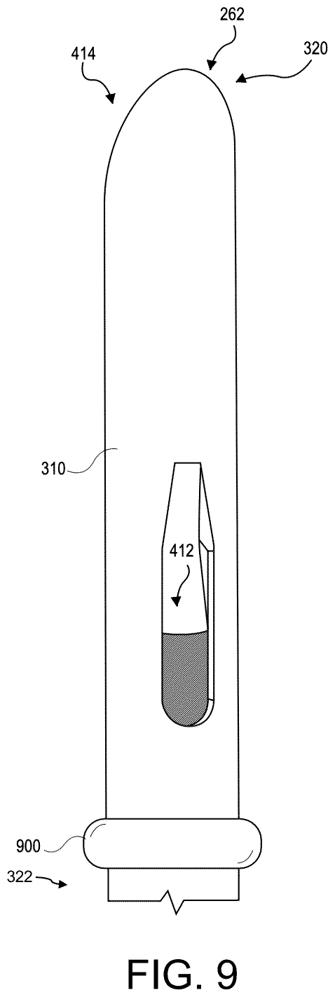

FIG. 9 is an isometric view of the shaft 310 according to one or more embodiments. An O-ring 900 is disposed on the shaft 310. The O-ring 900 is configured to mechanically engage the IV fluid bag to reduce water leakage around the shaft 310 and the dual-channel spike 260, such as by improving the fluid seal between the dual-channel spike 260 and the IV fluid bag. The O-ring 900 can be located at or towards the proximal end 322 of the shaft 310 (e.g., closer to the proximal end 322 than the distal end 320). In some embodiments, the shaft 310 can include a barb 800 and an O-ring 900.

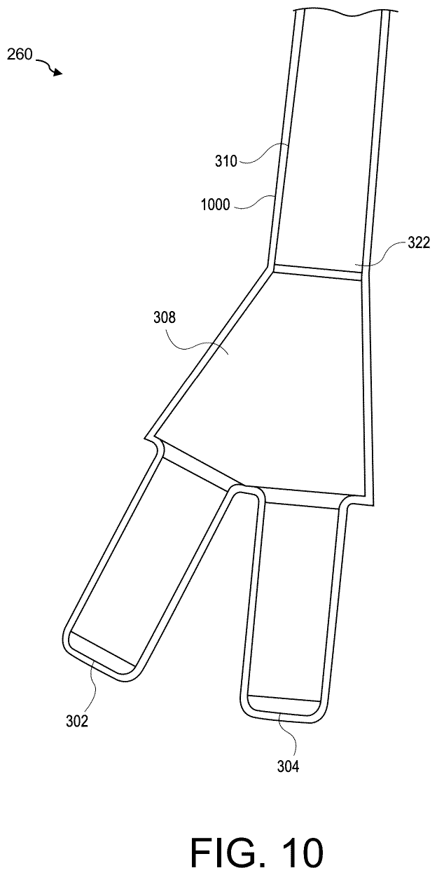

FIG. 10 is a side view of a proximal portion of the dual-channel spike 260 according to one or more embodiments. An over-mold 1000 is disposed on the dual-channel spike 260. The over-mold 1000 is configured to improve the fluid seal between the dual-channel spike 260 and the IV fluid bag. The over-mold 1000 can include a soft material, such as rubber or silicone, molded over a hard core of material that can allow the allow the dual-channel spike 260 to deform and/or conform to a spike port in an IV fluid bag 240 to improve the fluid seal between the dual-channel spike 260 and the spike port. The dual-channel spike 260 can include the over-mold 1000 in addition to or instead of the barb 800 and/or the O-ring 900. Though only a proximal portion is illustrated in FIG. 10, the over-mold 1000 can be disposed on all or substantially all of the dual-channel spike 260.

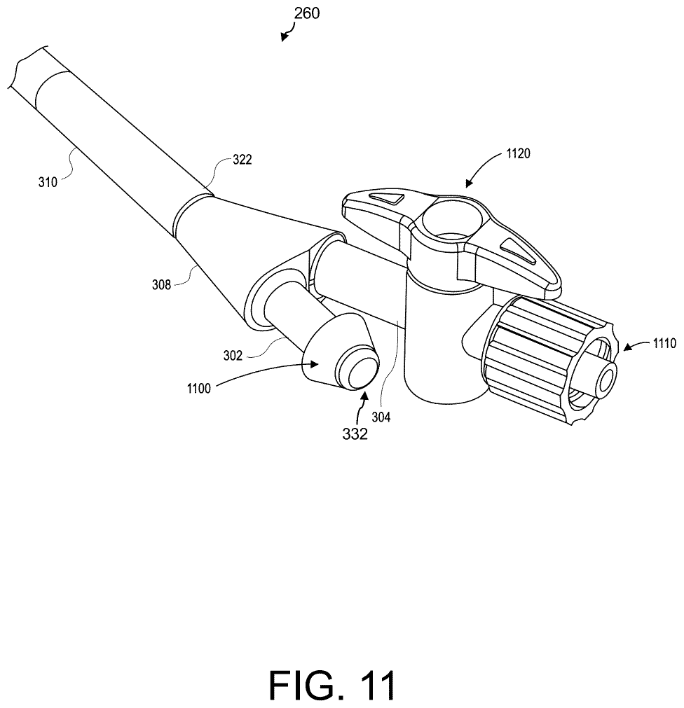

FIG. 11 is an isometric perspective view of a proximal portion of the dual-channel spike 260 according to one or more embodiments. A barb 1100 can be disposed on the outlet tube 302. The barb 1100 can be used to mechanically hold or couple the outlet tube 302 to another tube, such as an inlet line 242, and to provide a quick connection/disconnection.

In addition or in the alternative, a Luer fitting 1110 can be disposed at the end of the inlet tube 304 to form a Luer-lock connection with a corresponding Luer fitting on a tube (e.g., on an outlet line 244). The Luer fitting 1110 is a female Luer fitting but can be a male Luer fitting in another embodiment. The Luer-lock connection can provide a secure fluid connection between the inlet tube 304 and an outlet line 244.

In addition or in the alternative, a shut-off valve 1120 can be disposed on the inlet tube 304, such as between the Luer fitting 1110 and the handle 308. The shut-off valve 1120 can transition between an open state (as illustrated) and a closed stated.

In other embodiments, both the outlet tube 302 and the inlet tube 304 can include a respective barb 1100 and/or a respective shut-off valve 1120. Alternatively, both the outlet tube 302 and the inlet tube 304 can include a respective a Luer fitting 1110 and/or a respective shut-off valve 1120.

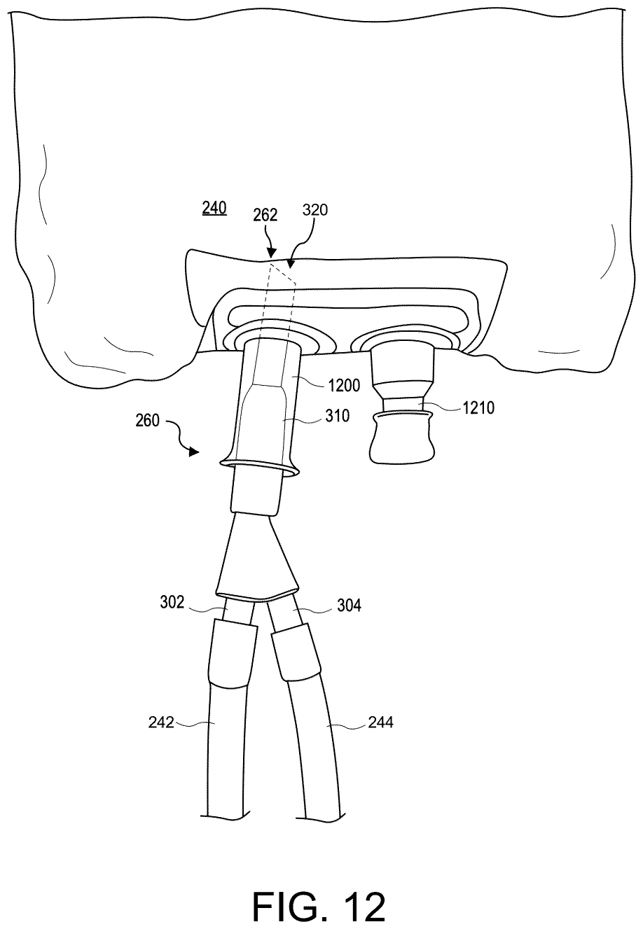

FIG. 12 shows an example dual-channel spike 260 inserted into an IV fluid bag 240 according to one or more embodiments. The dual-channel spike 260 in inserted in a spike port 1200 (e.g., by puncturing the spike port 1200 with the pointed end 262) at the bottom of the IV fluid bag 240 and adjacent to a standard fluid outlet tube 1210 in the IV fluid bag 240.

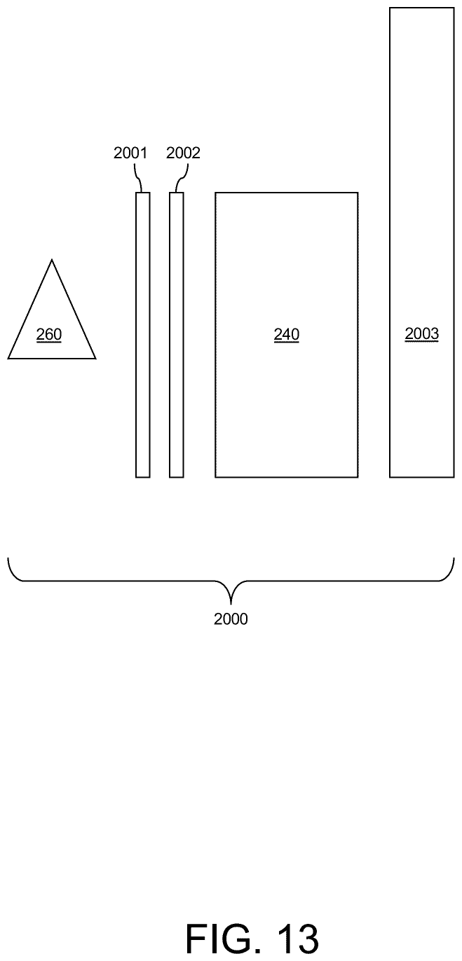

FIG. 13 is a block diagram of a kit 2000 according to one or more embodiments. In one or more embodiments, the kit 2000 includes at least a dual-channel spike 260 and first and second fluid lines 2001, 2002. The first fluid line 2001 is configured to fluidly couple a first opening (e.g., outlet opening 332) of the dual-channel spike 260 and an inlet of a cooling channel defined in a medical device. The second fluid line 2002 is configured to fluidly couple a second opening (e.g., inlet opening 334) of the dual-channel spike 260 and an outlet of the cooling channel defined in the medical device (e.g., in an ultrasound applicator 20 or in an ECD 1300). The first and second fluid lines 2001, 2002 can be the same as the inlet and outlet lines 242, 244, respectively. In one or more embodiments, the kit 2000 can further include an IV fluid bag 240 and/or a medical device 2003. The medical device 2003 can be or comprise an ultrasound applicator 20 or an ECD 1300. In one or more embodiments, the kit 2000 can include two or more (e.g., a plurality of) IV fluid bags 240.

In one or more alternative embodiments, the kit 2000 includes at least a dual-channel spike 260 and an IV fluid bag 240 (or multiple IV fluid bags 240). In one or more embodiments, the kit 2000 can further include a first fluid line 2001, a second fluid line 2002, and/or a medical device 2003.

In one or more alternative embodiments, the kit 2000 includes at least a dual-channel spike 260 and a medical device 2003. In one or more embodiments, the kit 2000 can further include an IV fluid bag 240 (or multiple IV fluid bags 240), a first fluid line 2001, and/or a second fluid line 2002.

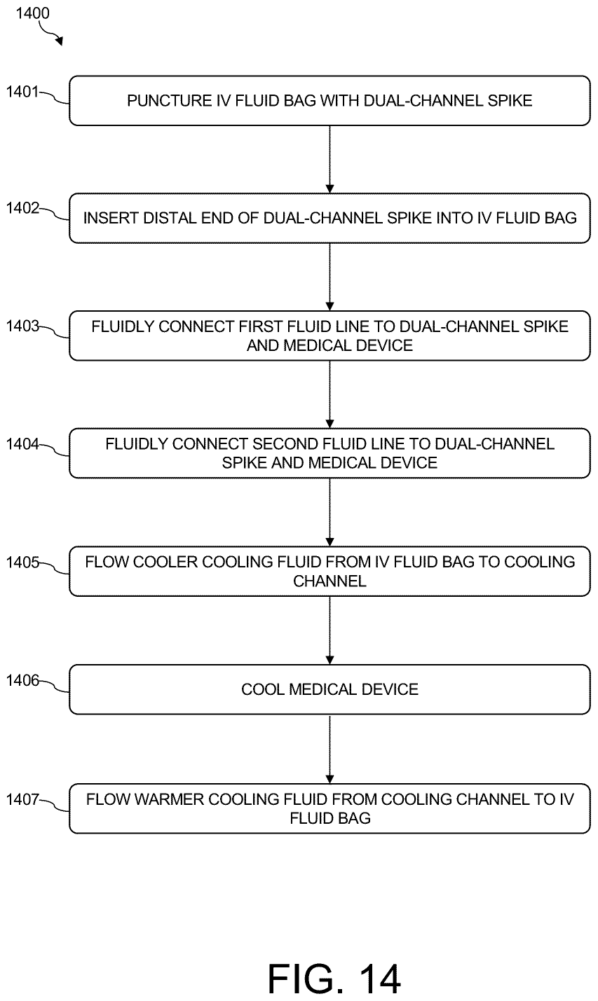

FIG. 14 is a flow chart of a method 1400 for recirculating cooling fluid using a dual-channel spike 260 according to one or more embodiments.

In step 1401, the pointed end 262 of the dual-channel spike 260 is used to poke or puncture a hole in an IV fluid bag 240. The hole can be formed in a spike port 1200 of the IV fluid bag 240.

In step 1402, the distal end of the dual-channel spike 260 (e.g., the distal end 320 of the shaft 310) is inserted through the hole formed in the IV fluid bag 240.

In step 1403, a first fluid line is fluidly coupled and/or connected to a first opening (e.g., outlet opening 332) of the dual-channel spike 260 and an inlet of a cooling channel defined in a medical device. The first fluid line can be the same as an inlet line 242. The medical device can be the same as the medical device 2003.

In step 1404, a second fluid line is fluidly coupled and/or connected to a first opening (e.g., outlet opening 332) of the dual-channel spike 260 and an outlet of the cooling channel defined in the medical device. The second fluid line can be the same as an outlet line 244.

In step 1405, relatively cooler cooling fluid flows from the IV fluid bag 240 to the cooling channel in the medical device. The cooler cooling fluid flows from the IV fluid bag 240 through a first port (e.g., an inlet port 412), a first channel (e.g., an outlet channel 402), and a first opening (e.g., an outlet opening 332) in the dual-channel spike 240 and through the first fluid line to the inlet of the medical device.

In step 1406, the cooler cooling fluid flows through the cooling channel and cools the medical device (e.g., by absorbing heat energy from the medical device) which causes the temperature of the cooling fluid to increase.

In step 1407, the warmer cooling fluid (e.g., that has warmed as a result of cooling the medical device) flows from the outlet of the medical device through the second fluid line, and through a second opening (e.g., an inlet opening 334), a second channel (e.g., an inlet channel 404), and a second port (e.g., an outlet port 414) in the dual-channel spike 240 and into the IV fluid bag 240

The warmer cooling fluid cools in the IV fluid bag 240 and can be recirculated to the cooling channel of the medical device (e.g., in the same manner as in step 1405).

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A medical system comprising:
a medical device having a cooling channel defined therein;
an intravenous (IV) fluid bag holding a fluid;
a dual-channel spike disposed in the IV fluid bag, the dual-channel spike defining first and second channels that are fluidly connected to the IV fluid bag, the dual-channel spike having a pointed distal end configured to puncture the IV fluid bag;
a first line fluidly connected to the first channel and to an inlet of the cooling channel; and
a second line fluidly connected to the second channel and to an outlet of the cooling channel,
whereby the fluid can be recirculated between the IV fluid bag and the cooling channel using the first and second channels in the dual-channel spike.

2. The medical system of claim 1, further comprising:
a first pump fluidly coupled to the first line; and/or
a second pump fluidly coupled to the second line.

3. The medical system of claim 1, wherein the dual-channel spike includes:
a shaft having proximal and distal ends,
first and second tubes attached to the proximal end of the shaft,
a first opening defined in the shaft, and
a second opening defined in the shaft,
wherein:
the first line is fluidly connected to the first tube,
the second line is fluidly connected to the second tube,
the first channel is fluidly coupled to the first opening and the first tube, and
the second channel is fluidly coupled to the second opening and the second tube.

4. The medical system of claim 3, wherein the first and second openings are positionally offset with respect to an axis of the shaft, the shaft extending along the axis.

5. The medical system of claim 4, wherein the first and second openings are offset with respect to a circumference of the shaft.

6. The medical system of claim 4, wherein the second opening is defined at or in the distal end of the shaft.

7. The medical system of claim 6, wherein the pointed distal end is located at the distal end of the shaft.

8. The medical system of claim 1, wherein the medical device comprises an ultrasound applicator or an endorectal cooling device.

9. A dual-channel spike comprising:
a shaft having inlet and outlet openings defined therein, a pointed distal end, and a proximal end, the shaft extending along an axis between the proximal end and the pointed distal end, the pointed distal end configured to puncture an intravenous fluid bag;
a handle attached to the proximal end of the shaft;
an inlet connector fluidly coupled to the handle, the inlet connector configured to be fluidly connected to a first fluid line;
an outlet connector fluidly coupled to the handle, the outlet connector configured to be fluidly connected to a second fluid line;
an inlet channel defined in the shaft and the handle, the inlet channel fluidly coupling the inlet connector and the outlet opening; and
an outlet channel defined in the shaft and the handle, the outlet channel fluidly coupling the outlet connector and the inlet opening.

10. The dual-channel spike of claim 9, wherein the inlet and outlet openings are positionally offset along the shaft with respect to the axis.

11. The dual-channel spike of claim 10, wherein the inlet opening is closer to the handle than the outlet opening.

12. The dual-channel spike of claim 11, wherein the inlet and outlet openings are offset with respect to a circumference of the shaft.

13. The dual-channel spike of claim 12, wherein the outlet opening is defined at or in the pointed distal end of the shaft.

14. The dual-channel spike of claim 9, wherein:
the axis is a first axis,
the shaft has a width that is measured with respect to a second axis that is orthogonal to the first axis, and
the pointed distal end has an angled planar face that is defined by a plane, the plane defined by (a) a third axis that is orthogonal to the first and second axes and (b) a fourth axis that is disposed at an angle relative to the first axis, the angle greater than or equal to about 30 degrees and less than or equal to about 80 degrees.

15. The dual-channel spike of claim 10, further comprising inlet and outlet tubes attached to a proximal end of the handle, the inlet and outlet connectors defined in the inlet and outlet tubes, respectively.

16. The dual-channel spike of claim 15, wherein:
the axis is a first axis,
the inlet tube extends along a second axis,
the outlet tube extends along a third axis, and
the second axis is disposed at an angle relative to the third axis, the angle greater than or equal to about 15 degrees and less than or equal to about 45 degrees.

17. The dual-channel spike of claim 9, wherein:
the axis is a first axis, and
a distal end of the handle has a smaller width than the proximal end of the handle, the width measured with respect to a second axis that is orthogonal to the first axis.

18. The dual-channel spike of claim 9, further comprising an over-mold covering at least an external surface of the shaft.

19. The dual-channel spike of claim 9, further comprising a barb or an O-ring disposed on the shaft, the barb or the O-ring configured to mechanically engage the IV fluid bag to improve a fluid seal between the dual-channel spike and the IV fluid bag.

20. A kit comprising:
an intravenous (IV) fluid bag; and
a dual-channel spike comprising:
a shaft having inlet and outlet openings defined therein, a pointed distal end, and a proximal end, the shaft extending along an axis between the proximal end and the pointed distal end, the pointed distal end configured to puncture the IV fluid bag;
a handle attached to the proximal end of the shaft;
an inlet connector fluidly coupled to the handle, the inlet connector configured to be fluidly connected to a first fluid line;
an outlet connector fluidly coupled to the handle, the outlet connector configured to be fluidly connected to a second fluid line;
an inlet outlet channel defined in the shaft and the handle, the inlet channel fluidly coupling the inlet connector and the outlet opening; and
an outlet channel defined in the shaft and the handle, the outlet channel fluidly coupling the outlet connector and the inlet opening.
